(19) United States
(12) Patent Application Publication
(10) Pub. No.: US 2026/0355770 A1
(43) Pub. Date: Nov. 12, 2026

(54) VALVE WITH BYPASS PATHWAY AND CORRESPONDING FILLING METHOD

(71) Applicant: AURENA LABORATORIES HOLDING AB, Karlstad (SE)

(72) Inventors: Björn Sörensen, Stockholm (SE); Barry Franks, Glemmingebro (SE)

(73) Assignee: AURENA LABORATORIES HOLDING AB, Karlstad (SE)

(21) Appl. No.: 19/134,897

(22) PCT Filed: Nov. 30, 2023
(86) PCT No.: PCT/EP2023/083829

§ 371 Date: Jun. 2, 2025

(30) Foreign Application Priority Data
Dec. 2, 2022 (SE) 2251411-1

(51) Int. Cl. B05B 12/08; B65D 83/42; B65D 83/52

(52) U.S. Cl. CPC B05B 12/088; B65D 83/425; B65D 83/52

(57) ABSTRACT

The invention relates to valve assemblies (400) for the dispensing of pressurised fluid comprising a valve body (402) with a metering chamber (422) and a pressure regulating valve (460) connectable to a vessel with fluid to be dispensed wherein the valve assembly further comprises a bypass pathway (458) extending past the metering chamber to the vessel, wherein said bypass pathway comprises a flow control arrangement (460) which allows one-way fluid flow from the surroundings in the direction towards and into the vessel.

### TECHNICAL FIELD

The present invention relates generally to valves assemblies, in particular valve assemblies for spray devices intended to dispense a measured volume of a product concentrate. Such spray devices include spray devices which are intended to provide a metered dose of a product to the mouth of a user. Such oral spray devices include those commonly known as metered dose spray devices (MDIs), which are intended to produce an aerosol of fine particles or droplets for inhalation through the mouth of the user for subsequent deposition in the pulmonary tract. The invention also relates to bag on valve (BoV) spray devices which provide metered doses of a product concentrate to a user in the field of nasal sprays, skin care sprays and the like.

### BACKGROUND

There are two well-known systems used in handheld spray devices to provide the propulsion force necessary to produce fine particle or droplets and propel them into the user's mouth or nose or onto the skin, namely the pressurised premetered MDI (pMDI) and bag on valve (BoV) systems. The pMDI system comprises an aerosol vessel which contains the aerosol formulation which has two components-the product concentrate and the propellant.

The aerosol vessel is a disposable container which comprises a, usually crimped-on, valve. Filing of the vessel with the product concentrate and propellant takes place though the valve in pMDI systems. In current BOV systems the bag is attached to the valve and then placed inside the vessel, initially with a gap maintained between the valve and the vessel opening. Compressed gas is injected through this gap until it reaches a first elevated pressure and then valve is crimped onto the vessel. The filing of the bag with the product concentrate can subsequently take place by reverse flow through the valve and metering chamber—this causes the bag to expand and increases the pressure in the vessel to a higher elevated pressure.

The product concentrate comprises the active ingredient(s)of the aerosol such one or more pharmaceutical preparations, combined as necessary with any additives such as antioxidants, surfactants, and solvents which are required to prepare a stable and effective product concentrate.

The propellant can be one of two types: i) a liquefied gas or a mixture of liquefied propellant gasses that may serve a dual role as both the propellant and the solvent or vehicle for the product concentrate, or ii) compressed gas propellants. Actuation of an actuator moves a valve stem into the metering chamber which causes an opening in the valve stem to communicate with the metering chamber where a pre-metered dose of the product concentrate and propellant in pMDI systems is stored while at the same time the end of the valve step acts on a valve seat between the metering chamber and the vessel to prevent more product concentrate and propellant from entering the metering chamber. This pre-metered dose exits the dosing chamber via the valve stem and is dispensed, usually though a mouthpiece in the case of oral spray devices, to the user. Releasing the actuator causes the valve stem to move away from its valve seat. This has two effects-its initial movement moves the opening in the valve stem out of the metering chamber, thereby prevent further dispensing of the contents of the metering chamber while subsequent movement moves the end of the valve stem off the valve seat thereby allowing more product concentrate and propellant to enter the metering chamber.

Commonly used liquefied propellant gasses are chlorofluorocarbons (CFCs) such as dichlorodifluoromethane, dichlorotetrafluoroethane and trichloromonofluoromethane. When a liquefied gas propellant is sealed within an aerosol vessel with the product concentrate, equilibrium is quickly established between portion of the propellant that remains liquefied and that which vaporizes and occupies the upper portion of the aerosol vessel. The vapor phase exerts pressure in all directions against the walls of the vessel, the valve assembly and the surface of the liquid gas which is composed of the liquefied gas and the product concentrate. It is this pressure that after actuation of the dispensing valve, forces the liquid phase up a dip tube into the newly emptied metering chamber for subsequent release out of the valve stem and out of the orifice of the valve into the atmosphere. As the propellant meets the air, it expands and evaporates because of the drop in pressure, leaving the product concentrate as airborne liquid droplets or dry particles depending on the formulation. As the liquid phase is removed from the vessel, equilibrium between the propellant remaining liquefied and that in the vapor state is re-established by vaporization of the liquid phase. These provide a substantially constant delivery pressure until the liquid phase has been exhausted, after which the pressure drops after each activation of the spray device.

Compressed gas propellants include nitrogen, nitrous oxide and carbon dioxide. When used in a bag-on-valve system the product concentrate is contained in a flexible bag which is connected to the inlet of the metering chamber and valve assembly, and the propellant is provided in the space between the walls of the aerosol vessel and the bag. The propellant exerts a pressure on the flexible bag. When the valve is activated by the user, for example by pressing an actuator connected to the valve stem, then, in a manner similar to that described above with respect to the pMDI, a valve stem is moved to a position which allows dispensing of the contents of the metering chamber while preventing refilling of the metering chamber until the valve actuator is released. It is the pressure exerted by the pressurized gas which causes some of the contents of the bag to be expelled though the valve upon each actuation of the actuator. The pressurised gas normally never leaves the vessel but each time the spray device is actuated the pressure of the pressurised gas drops. This means that the delivery pressure falls each time the spray device is activated. As normally a minimum delivery pressure is necessary to give the desired droplet profile, after a predetermined number of activations such spray devices are usually deemed to be unusable because even though such compressed gas remains in the space between the bag and the vessel internal wall, the pressure of the gas is too low to guarantee the correct droplet distribution and dispensing plume.

### PROBLEMS WITH THE PRIOR ART

A problem with liquified propellant gases is that these gasses are environmentally unfriendly and the use of them is being curtailed or banned. In the future it will not be permitted to use them as propellants which are released to the atmosphere. Thus, it will become necessary to use an environmentally friendly propellant which, of course, is also not harmful to a user in pMDI systems, for example a compressed gas. A problem with compressed gas propellants, which are often used in BoV systems, is that the delivery pressure falls as the spray device is used. As the dispersion pattern and size of the droplets generated during actuation is dependent on the delivery pressure of the propellant, the spray devices become unable to deliver the required droplet pattern and size if the delivery pressure falls too much. This means that the device may stop working before all the product concentrate has been used up. This is wasteful and costly.

U.S. Pat. No. 9,364,620B2 teaches a device for providing medication nasally which uses a removable gas cylinder with high pressure gas, two pressure regulators and a refillable medication supply. It requires two regulators to reduce the high pressure in the gas cylinder and to prevent the loss of control of the dispensing pressure which would occur in a single stage regulator if the supply pressure in the gas cylinder became too low. It is complicated and expensive to construct. Furthermore, the valve system is large and is provided as a separate unit to which a separate supply of medication and a sperate prefilled gas cylinder can be attached. This is not suitable for use with handheld spray devices which provide the medication orally.

There is thus a need for an improved spray device valve which provides the required delivery pressure for a large number of actuations while avoiding the use of environmentally harmful propellants. Preferably such a valve system may be applicable both to pressured metered dose spray devices and bag on valve systems using compressed gases.

In order to fulfil current and future requirement a valve assembly for a handheld spray device for dispensing a product concentrate preferably should be:
i) compact in size so that it is compatible with the dimensions of contemporary filling machines and contemporary spray devices and spray device actuating mechanism,
ii) attachable to a compressed gas vessel,
iii) able to allow filling of a gas vessel with pressurised gas and, if appropriate, active substances via the valve assembly after the valve assembly has been attached to a compressed gas vessel;
iv) suitable for use with pressurised gas propellants to give a substantially constant delivery pressure during the required dispensing life of the vessel to which it is attached,
v) inexpensive to manufacture.

### SUMMARY

The above-described problem is addressed by the claimed valve assembly. These valves have the features of claim 1. A vessel and valve assembly have the features of claim 6. A method for filling such a vessel and valve assembly has the features of claim 9.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly. Relative positional terms such as up, down, above, below are merely used in reference to the orientations shown in the figures and are not intended to limit the scope of protection of the valve assemblies of the invention which may be used in orientations which are different to those shown in the figures,

### BRIEF DESCRIPTION OF THE DRAWINGS

FIG. 1 is a schematic cross-section illustrating the components of a prior art pMDI spray device.

FIG. 2 is a schematic cross-section illustrating the components of a prior art BoV spray device.

FIGS. 3a) and 3b) illustrate schematically cross-sections of a metering valve in the open and closed positions.

FIG. 4 is a schematic perspective view of a valve assembly in accordance with a first embodiment of the invention.

FIG. 5 is a schematic view of the valve assembly of FIG. 4 after it has been crimped to the neck of a vessel.

FIG. 6a) show schematically a cross-section though the valve assembly of FIG. 5 with the valve stem in a dispensing or filling position. FIG. 6b) shows schematically in perspective the valve stem from the valve assembly of FIG. 6.

FIG. 7 shows schematically the cross-section though the valve assembly of FIG. 6A with the valve stem in the metering chamber filling position.

FIG. 8a) shows schematically a cross-section of a second embodiment of the invention with the valve stem in a dispensing or filling position. FIG. 8b) shows schematically in perspective the valve stem from the valve assembly of FIG. 8a).

FIG. 9 show schematically a cross-section though a third embodiment of the invention, for use with a bag on valve system, with the valve stem in a dispensing or filling position.

FIG. 10 schematically illustrates a cross section through the valve assembly of FIG. 9 with the valve stem in the metering chamber filling position.

FIG. 11 show schematically a cross-section though a fourth embodiment of the invention, for use with a bag on valve system, with the valve stem in a dispensing or filling position.

FIG. 12 schematically illustrates a cross section through the valve assembly of FIG. 11 with the valve stem in the metering chamber filling position.

FIG. 13 illustrates the steps in a method for filing a vessel provided with a valve in accordance with the present invention.

FIG. 14a) to 14 c) show schematically in perspective and in cross-section a further embodiment of a BoV valve assembly according to the invention.

FIG. 15a) to 15 c) show schematically in perspective and in cross-section a further embodiment of a pMDI valve assembly according to the invention.

Embodiments of the present disclosure and their advantages are best understood by referring to the detailed description that follows. It should be appreciated that like reference numerals are used to identify like elements illustrated in one or more of the figures.

### DETAILED DESCRIPTION

The present disclosure relates generally to devices, systems and methods for dispensing valves and dispensing valve assemblies, in particular metered dispensing valves and assemblies using such valves for spray devices. In the following a valve stem is said to be open when it is placed in a position in which it provides fluid communication between the interior of the metering chamber and the surroundings-this can occur when the valve stem is depressed to allow dispensing from a metering chamber or to allow filing into a metering chamber. The valve stem is said to be closed when it prevents the passage of fluid from the metering chamber to atmosphere.

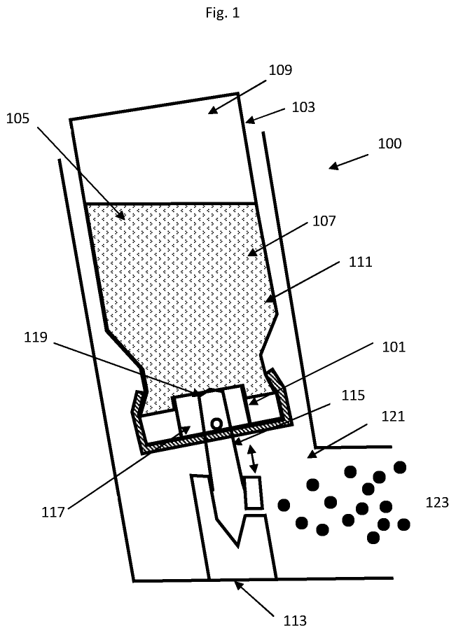

Embodiments of the disclosed solution are presented in more detail in connection with the figures. FIG. 1 schematically illustrates across-section through a prior art PMDI valve in a spray device 100. This comprises a metering valve 101 crimped onto a substantially rigid vessel 103 containing the product 105 in solution or suspension in a propellant 107. The product is kept under pressure by the vapourised gas phase 109 of the propellant which is formed from the liquid phase 111 of the propellant in the vessel. When an actuator 113 is moved upwards a valve stem 115 is opened to dispense fluid from the metering chamber 117, while at the same time the valve stem moves to a sealing position which prevents more of the liquid phase propellant and product from entering the metering chamber 117 via an opening 119. The fluid in the form of pressurised propellant and product in the metering chamber leaves the metering chamber and enters the expansion chamber 121 where they are subsequently dispensed as a high velocity spray 123.

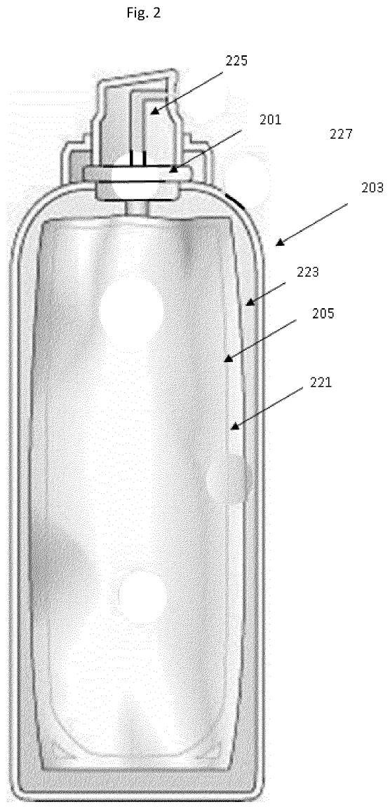

FIG. 2 schematically illustrates a prior art bag on valve (BOV) valve assembly on a vessel 203. This comprises a metering valve 201 and a flexible bag 221 containing the product 205. The product is kept under pressure by pressurized gas propellant 223 contained between the bag 221 and the inner wall 223 of the vessel 203. The product is dispensed by pressing the head 225.

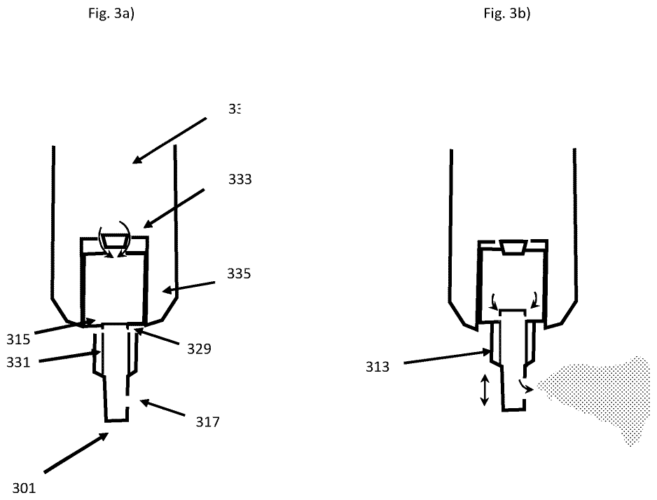

FIGS. 3a) and 3b) illustrate the principles of one type of prior art metering valve 301. As shown in FIG. 3a), in the closed position the inlet openings 329 in the valve stem 331 are not inside the metering chamber 315 (thereby preventing the contents of the metering chamber from being in communication with the nozzle 317) and the inlet valve 333 between the metering chamber 315 and the interior 335 of the pressurized vessel 303 is open (thereby allowing the metering chamber 315 to be filled with pressurized propellant and product from inside the vessel 303).

As shown in FIG. 3b), when the actuator 313 is moved towards the vessel then the metering chamber is first pushed into sealing engagement against the inlet valve 333 and then subsequently the metering chamber 315 is connected to atmosphere, i.e., as the metering valve 301 moves to the open/dispensing position, the inlet openings 329 in the hollow valve stem 331 are moved to a position inside the metering chamber 315 and, since the pressurized contents of the metering chamber are thereby brought into fluid communication with the surrounding atmosphere, the contents of the metering valve can be discharged through the valve stem. At the same time, the inlet valve 333 between the vessel 309 and metering chamber 315 has been closed by the movement of the metering chamber towards the vessel, hereby preventing any flow between the interior 335 of the vessel 309 and the metering chamber 315.

After the contents of the metering chamber have been dispensed, the actuator is released, the valve stem returns to the closed position shown in FIG. 3a), blocking fluid communication of the metering chamber to atmosphere but allowing flow from the interior of the vessel to the metering chamber until the pressure in the metering chamber and the vessel are equal, thereby refilling the metering with a measured dose of pressurised product.

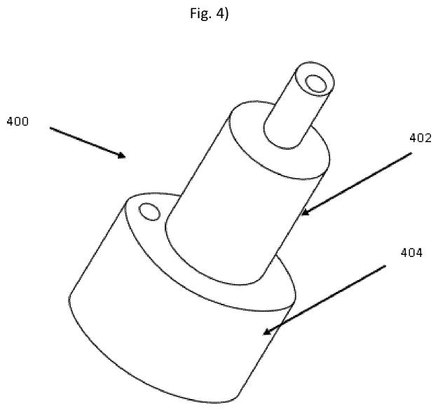

FIG. 4 is a schematic perspective view of a first embodiment of a valve assembly 400 in accordance with the present invention before it is attached to a vessel.

The valve assembly comprises a valve body 402 which is sealingly attached to a surrounding conventional crimping cap 404 which can be crimped in the conventional manner onto the neck of a vessel (not shown) which will contain the propellant and product.

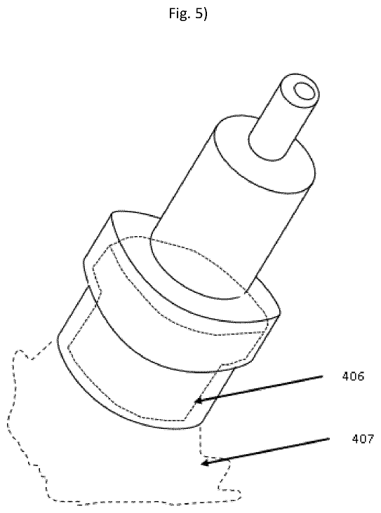

FIG. 5 is a schematic view of the valve assembly of FIG. 4 after it has been crimped to the neck 406 of a vessel 407 (only partly shown by dashed lines).

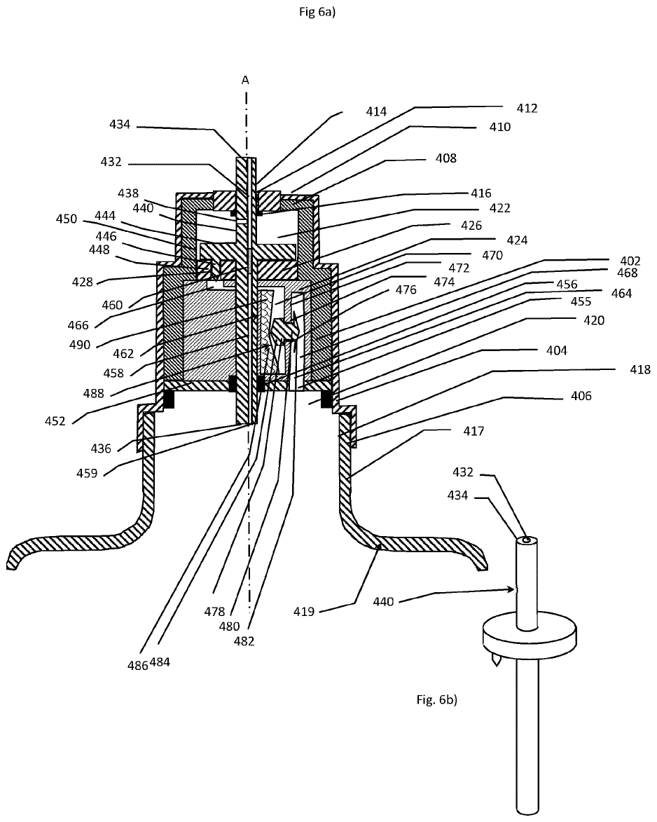

FIG. 6a) is a schematic cross-sectional view through the centreline of the valve assembly 400 of FIG. 4. The valve assembly comprises a valve body 402 which is hollow and cylindrical and has a first end 408 and a second end 418. The first end 408, which is visible during use, is covered by an end cap 410 which has an opening 412 to receive a valve stem 414. The opening 412 is sealed by a valve body to atmosphere seal 416 which allows the valve stem to slide in and out of the opening 412 without any leakage between the valve stem and the opening. During use, the opposite, second end 418 of the valve body is intended to be attached to, or inside, the neck 417 of a, preferably rigid, vessel 419 such as a canister, bottle or bag or the like (shown partly by dashed lines) which encloses the product that is to be dispensed. The valve assembly is sealingly attached to the neck 417 of the vessel and has an opening 420 to allow the passage of the product from the vessel to the interior of the valve body. The valve body is divided into a metering chamber 422 and a pressure regulating portion 424 by an interior partition wall 426 which prevents uncontrolled fluid flow from the pressure regulating portion to, helps to define the volume of, the metering chamber. Preferably the partition wall extends across the interior of the valve body, preferably perpendicular to the longitudinal centreline axis A of the valve body. The metering chamber has a metering volume (Vm) which is the free volume available for the dose being. For a valve used for a spray device the metering volume Vm is typically between 0.025 ml to 1.0 ml. Preferably the metering volume is equal to or greater than 0.05 ml and equal to or less than 0.25 ml.

The pressure regulating portion 424 comprises a pressure regulating valve 462. The pressure comprises a high-pressure inlet 464 which is in fluid communication with the pressurised gas and product containing vessel 419 and a regulated pressure outlet 466 which is in sealable fluid communication with the metering chamber. This can be achieved by the partition wall 426 comprising at least one closable fluid dispensing through hole 428. Preferably the partition wall comprises one or a plurality of fluid dispensing through holes 428 which, when unobstructed, allow fluid communication between the pressure regulating portion and the metering chamber. The pressure regulating valve comprises an inlet chamber 468 in fluid communication with high-pressure inlet 464 and a regulated pressure outlet chamber 470 in fluid communication with fluid dispensing through hole 428. The inlet and outlet chamber are separated by an intermediate chamber wall 472 with a through hole 474. The through hole 474 has a valve surface 476 facing the interior of the high-pressure chamber. The through hole accommodates a poppet valve 478 which has a poppet valve stem 480 which extends through the through hole 474 and has a valve head 482 in the high-pressure inlet chamber which is adapted to seal against the valve surface 476. The opposite actuating end 484 of the poppet valve stem is in the outlet chamber and is joined to or in contact with a pressure regulating diaphragm or surface 486 which extends across the outlet chamber and separates it from a pressure regulating chamber unit 488. The pressure regulating chamber comprises poppet valve biasing means 490 which bias the poppet valve in the direction towards the high-pressure inlet chamber. The biasing means 490 push the poppet valve open with a force which corresponds to the desired metering valve chamber pressure. The biasing means can be any appropriates force producing means. It could be in the form of a compression spring, for example a helical spring or a Belleville spring or resilient material, force-producing arms acting on the diaphragm or surface 486, or the pressure regulating chamber could be a sealed chamber containing a gas which generates a force which gives the desired biasing force. The diaphragm could itself be shaped to produce the desired biasing force, for example by being concave towards the poppet valve. The biasing means could be formed by a block of rubber-like, gel or sponge-like resilient material which is deformable when pushed on by the poppet valve and resists movement of the actuating end of the poppet valve when the poppet valve is pushed towards it. The amount of resistance can be changed by, for example, changing the surface area of the contact surface of the actuating end of the poppet valve which is in contact with the resilient material.

When the supply pressure from the compressed gas in the vessel becomes too low to overcome the biasing force holding the valve open then all the remaining compressed gas and product concentrate in the vessel is free to pass through the open valve opening. However, the presence of a metering chamber prevents this fluid from flowing directly to atmosphere-instead it fills the metering chamber until the pressure in the metering chamber is in equilibrium with the pressure in the vessel. This leads to a “gentle” end of life of the dispensing system as it is still able to dispense measured doses of fluid, albeit at gradually reducing pressures as the propellant becomes exhausted, even when the pressure of the compressed gas is below the regulated pressure.

When the pressure regulating valve is in use, the high-pressure fluid (i.e. propellant gas and product concentrate) from the vessel can flow though the through hole and push on the diaphragm. As the force on the diaphragm overcomes the force provided by the biasing means 490 the poppet valve moves towards the valve seat until it reaches the closed position thereby preventing more high-pressure fluid from entering the outlet chamber. This limits the pressure in the outlet chamber to that determined by the biasing force acting on the diaphragm. This pressure is maintained in the outlet chamber while the fluid dispensing though holes are obstructed as described below. When the fluid dispensing through hole(s) are unobstructed, fluid can flow through them, thereby lowering the pressure in the outlet chamber which allows the biasing means to push the poppet valve towards the high-pressure chamber, thus once again allowing high-pressure fluid to enter the outlet chamber until the force of the biasing means is overcome and the poppet valve closed again.

The valve stem 414 has a longitudinal bore 432 which extends from a first end 434 of the valve stem which extends outside of the valve body and a second end 436 of the valve stem which is in fluid connection with the interior of the vessel which contains the product which is to be dispensed. The valve stem has a transverse bore 438 which leads from outer surface 440 of the valve stem to the longitudinal bore 432.

As is conventional, the valve stem can be moved longitudinally between a closed position in which the fluid dispensing through holes are obstructed, thereby preventing pressurised fluid flow into the metering chamber and an open position in which the through holes are not obstructed and allow the passage of pressurised fluid into the metering chamber from the pressure regulating portion. As an example, the fluid dispensing through holes may be obstructed and sealed by a radial enlargement 444 of the valve stem 414 with an appropriate number of corresponding sealing projections 446 which, when brought near to or into contact with the partition wall, project into, and sealingly contact the wall(s) 448 of the through hole(s), thereby sealing the through hole(s) 428. The tips of the sealing projections could be made of a resilient material such as rubber or silicon or the like to ensure good sealing. The transverse bore 438 is positioned so that when the valve stem is in the open position (which is the resting position where dispensing of the product does not take place) the transverse bore is not in fluid communication with the metering chamber, thereby preventing fluid from exiting the metering chamber via the valve stem, and when the valve stem is in the closed position (which is the active position where dispensing of the product can take place) the transverse bore is fluid communication with metering chamber, thereby allowing the contents of the metering chamber to exit to atmosphere via central bore and the open first end of the valve stem. In order to allow fluid flow from the pressure regulating portion to the metering chamber, the peripheral surface 450 of the radial enlargement of the valve stem is not in sealing contact with the metering chamber wall and/or has appropriately placed through holes (not shown) which allow fluid flow through it or past it.

The pressure regulating portion comprises a pressure regulating valve 462 which is adapted to reduce the pressure of any fluid passing though it from the vessel to the metering chamber to the pressure needed to dispense the product. The pressure regulating portion is supported on, or comprises, a transversing wall 452 which extends in any suitable manner across the cross section of the valve body. Said transversing wall 452 does not have to be perpendicular to the longitudinal axis of the valve body but may be inclined to said axis, nor does said transversing wall have to be flat—it may comprise different levels or steps as it extends from one side of the valve body to the opposite side. The transversing wall prevents any fluid communication between opposite sides of the transversing wall except through specifically arranged passages and through holes. The transversing wall 454 has an inlet passage 455 which is open to the interior of the vessel to which the valve assembly can be fitted and leads to the inlet of pressure regulating valve.

The valve stem penetrates the transversing wall 452 and opens out into the vessel. A seal 456 is provided between the valve stem and the transversing wall to prevent leaks.

Conventional filling of a vessel using a valve assembly according to the present invention is not possible as reverse flow through the valve stem and metering chamber is prevented by the poppet valve or the like in the pressure regulating portion. Thus, in order to allow a vessel or bag within a vessel to which the valve assembly is attached to be filled while at the same time preventing any contents of the vessel, such as pre-filled product concentrate, from escaping, the valve assembly is provided with a bypass pathway 458 which leads from the exterior of the valve body to a position in fluid communication with the interior of the vessel or bag within the vessel (called vessel for brevity in the following) which is to be filled. The bypass pathway 458 provides a route for filling a vessel wherein the route bypasses the pressure regulating valve. The bypass pathway can be formed as required, for example, as shown here, as the portion of the central bore which is between the transverse bore 438 and an opening 459 in the second end 436 of the valve stem, and comprises a flow control valve 460 which can be any type of non-return valve., such as a spring-loaded ball valve or a duck-billed valve, which opens during filling of the vessel in order to allow the passage of fluid from the outside of the valve assembly via the bypass pathway to the interior of the vessel but which is normally closed in order to prevents the flow of fluid from the interior of the vessel to the exterior of the valve assembly. As a safety feature to avoid contamination by a high ambient pressure before filling of a vessel to which the valve assembly is attached the flow control valve preferably is set to open when it is exposed to an opening or cracking pressure PC which is higher than the dispensing pressure of the valve assembly and which is lower than the operating pressure Po which the vessel is intended to contain during use. Preferably the cracking pressure is greater than the dispensing pressure PD by 200 kPa or more. Alternatively, the flow arrangement could comprise a means for permanently sealing the bypass pathway after filling has taken place, for example a plug which could be placed in the bypass pathway to prevent flow though it and thereby prevent reuse of a valve assembly.

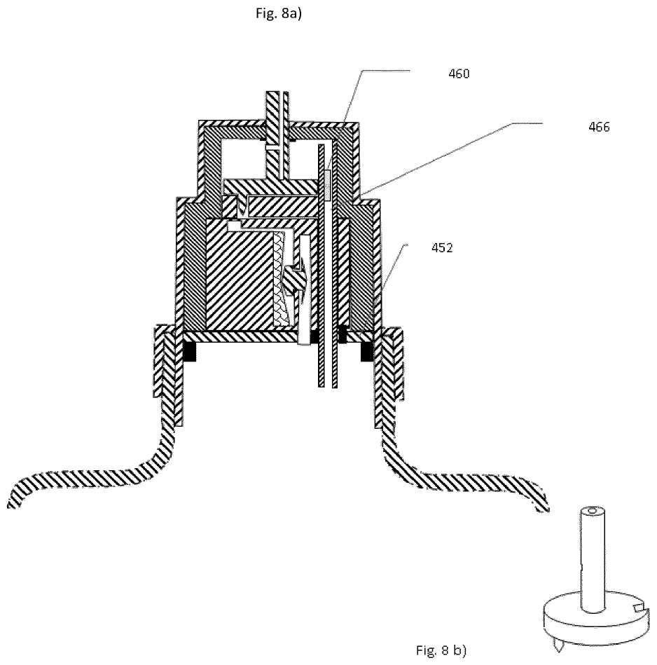

For use with conventional filling arrangements, a bypass pathway according to the present invention may be in the form of a channel which extends from the metering chamber through the partition wall and into the vessel as shown in FIG. 8 below. In such an embodiment of the present invention, the filling path would therefore be through, or beside, the valve stem into the metering chamber and then though the bypass pathway into the vessel.

For PMDI systems where the product to be dispensed is inside a rigid vessel then preferably the maximum dispensing pressure is 1600 kPa, the maximum cracking pressure of the flow control valve is 2500 kPa, the maximum operating pressure inside the rigid vessel is 2000 kPa after filling and the maximum filling pressure is 5000 kPa.

More preferably the minimum dispensing pressure is 500 kPa, the minimum cracking pressure of the flow control valve is 100 kPa. More preferably the minimum operating pressure inside the rigid vessel after filling is 600 kPa. More preferably the minimum filling pressure is 900 kPa.

Even more preferably the dispensing pressure is equal to or greater than 1350 kPa and less than or equal to 1600 kPa. Even more preferably the cracking pressure of the flow control valve equal to or greater than 100 kPa and less than or equal to 2500 kPa. Even more preferably the operating pressure inside the rigid vessel after filling is equal to or greater than 1500 kPa and less than or equal to 1800 kPa. Even more preferably the filling pressure is equal to or greater than 2500 kPa and less than or equal to 4500 kPa.

In the first embodiment of a valve assembly with a bypass pathway, as shown in FIG. 6, the valve stem extends directly into the vessel during use. As alternatives, the valve stem may be sliding engaged in a hollow sleeve which is itself in fluid connection with the interior of a vessel, or it may be in fluid connection to the interior of the vessel by some other means. In this embodiment of the invention the central bore of the valve stem forms the bypass pathway 458 which bypasses the pressure regulating chamber and allows the passage of fluid from the first end of the valve stem to the interior of the vessel only if the fluid is provided at a pressure which is above the opening pressure of the flow control valve 460.

FIG. 6A shows the first embodiment of the valve assembly of the present invention when the valve stem has been moved towards the valve body and the sealing projections 432 of the extension of the valve body have closed the fluid dispensing through holes. In this position the transverse bore of the valve stem is inside the metering chamber and the contents of the metering chamber are in fluid communication with the surroundings via the transverse bore and central bore of the valve stem. If the contents of the metering chamber are at a pressure which is higher than the surroundings then the contents of the metering chamber will be dispensed to the surroundings. As long as the pressure in the metering chamber is lower than the opening pressure of the non-return valve in the bypass pathway no fluid will flow from the metering chamber to the interior of the vessel.

When it is desired to fill the vessel to an operating pressure Po the open end of the valve stem can be connected to a source of high-pressure fluid at a filling pressure PF. The cracking pressure must be equal to or less than the filling pressure PF. Generally, the higher the difference between the filling pressure and the cracking pressure, the quicker the vessel can be filled. The filling pressure must at least be equal to or greater than the operating pressure. Preferably the filling pressure is greater than or equal to 25% greater than the operating pressure as this pressure differential can speed up the filling process. More preferably the filling pressure is greater than or equal to 50% greater than the operating pressure. In an embodiment of a method for filling a vessel via a valve body of the present invention, the time that the valve stem is open to the source of high-pressure fluid is controlled and limited so that the pressure in the vessel reaches the desired operating pressure without being over-pressurised if the filling pressure is close to or above the safe working pressure of the vessel.

When the valve stem is connected to the source of high-pressure fluid, the fluid at the filling pressure will enter the central bore of the valve stem. Once the pressure in central bore is greater than the cracking pressure of the flow control valve in the central bore then the flow control valve will open and allow the flow of fluid into the vessel. Once the filing time TF necessary for the pressure in the vessel to reach the operating pressure Po has elapsed, or if the pressure measured in the vessel is at the operating pressure, the source of high pressure is disconnected from the valve stem. This causes the pressure in the bypass pathway to drop below the cracking pressure as it falls to ambient pressure and the flow control valve will close, preventing fluid from exiting the vessel via the bypass pathway. In this embodiment of a bypass pathway, if the valve stem is move to the active position during filling, for example by being depressed by a filling head, then the pressure in the metering chamber will be raised to that of the filling pressure as the high-pressure fluid flows through the transverse bore into the metering chamber. This pressure will be released the first time that the valve stem is depressed after filling. Of course, it is not necessary to move the valve stem to the active position in order to fill a vessel as the function of the bypass pathway and the flow control valve is independent of the position of the valve stem—in such cases it is only necessary to move the valve stem far enough such that the transverse bore is blocked by the seal 426 or to cover the transverse bore by some sealing part of the filling head.

The pressure regulating valve is adapted to reduce the pressure inside the vessel to the dispensing pressure. After the vessel has been filled, it is preferable to active the valve stem once in order to purge the metering chamber of any high-pressure fluid which might have entered it during the filling operation. Subsequently, once the valve stem is moved to the active position where the fluid dispensing through holes are no longer obstructed by the projections on the valve stem, the fluid will flow from the vessel via the pressure regulating valve to the metering chamber until the pressure in the metering chamber reaches the dispensing pressure which is regulated by the pressure regulating valve. The valve and vessel assembly are then ready for use.

The valve is activated by a user to dispense the contents of the metering chamber by depressing valve stem towards the interior of the valve assembly. For example, this can be achieved by an actuator fitted to an actuator body of an inhaler or mask, or a simple push button attached to the valve stem. Once the valve stem has moved towards the partition wall sufficiently far enough so that the transverse bore of the valve stem enters the metering chamber, the pressurised contents of the metering chamber can immediately enter the bore of the valve stem and be released to atmosphere via the opening on the exposed end of the valve stem. Once the contents of the metering chamber have been released, the user releases the valve stem. As the valve stem is no longer being pushed towards the interior of the valve assembly by the user, the pressure of the fluid at the outlet of the pressure regulating valve will push the valve stem away from the partition wall so that the lateral opening of the valve stem is outside the metering chamber, thereby preventing any fluid from leaving the metering chamber and allowing the metering chamber to be filled with fluid at the dispensing pressure. I.e. releasing the valve stem takes away the force acting to hold the projections into contact with the partition wall, so the projections will be lifted away from the partition wall and allow fluid from the pressure regulating valve to enter the metering chamber until equilibrium is approached between the pressures in the metering chamber and the pressure regulating valve.

In this manner the metering chamber is refilled with fluid which is at a pressure determined by the pressure regulating valve.

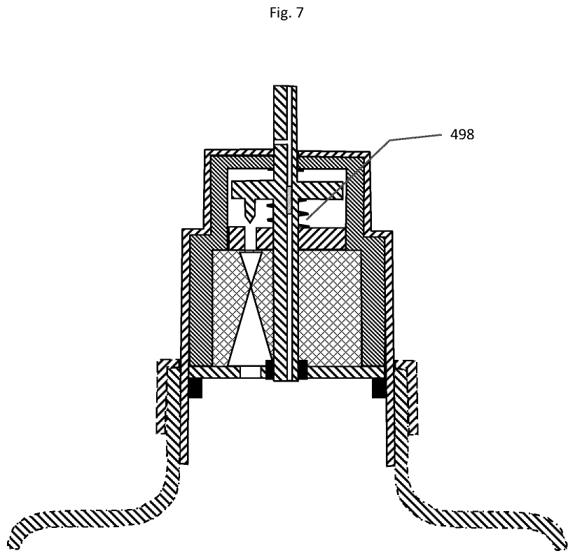

Optionally, a spring 498 (shown by dashed lines in FIG. 7) may be provided to bias the valve stem away from the partition wall and thereby assist the valve stem in returning from the active position.

It should be noted that a valve assembly using the above bypass arrangement of the valve stem can be used for filling a vessel which lacks a pressure regulating valve, in which case during use the metering chamber will always be filled with fluid at the same pressure as that which is in the vessel. FIG. 8a) shows a second embodiment of a valve assembly in accordance with the present invention. The same reference numbers are used for parts which are identical to or similar in function to that of the first embodiment of the invention. In this embodiment of the invention the valve stem does not contain a bypass pathway. The bypass pathway is formed as a channel 466 which extends from the metering chamber to, or through, the transversing wall 452. The channel contains a flow control valve 460 of the type described above which allows the flow of high-pressure fluid into the vessel but not from the vessel. Filing takes place through the valve stem, or as reversed flow outside the valve stem and past the valve stem seal, into the metering camber and then through the bypass pathway into the vessel or bag.

Preferably the channel has an axially extending exterior wall 468 which projects radially towards the centre of the valve body. The exterior wall preferably has a constant profile and the radial extension of the valve body is provided with a correspondingly shape groove which during use surrounds the profile which thereby forms a guide surface for the axial movement of the valve stem. This also helps to align the dispensing through hole sealing projections on the valve stem with the fluid dispensing through holes.

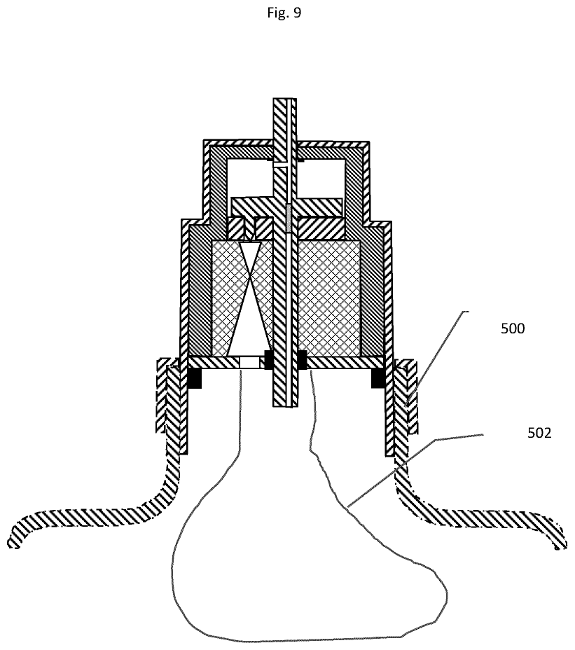

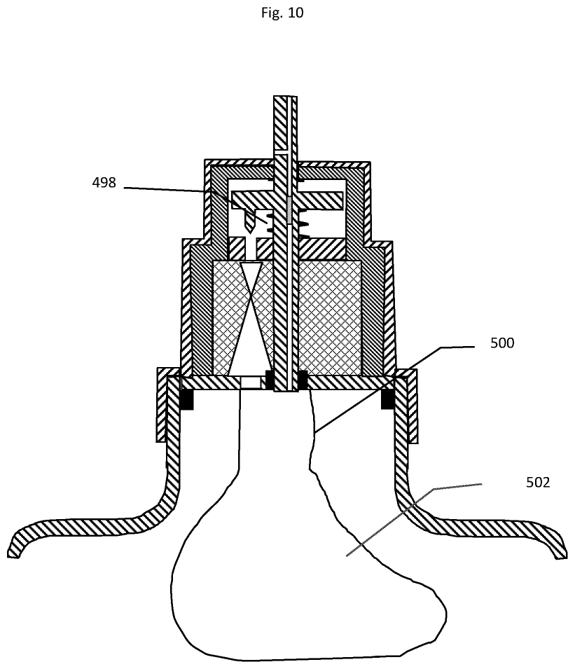

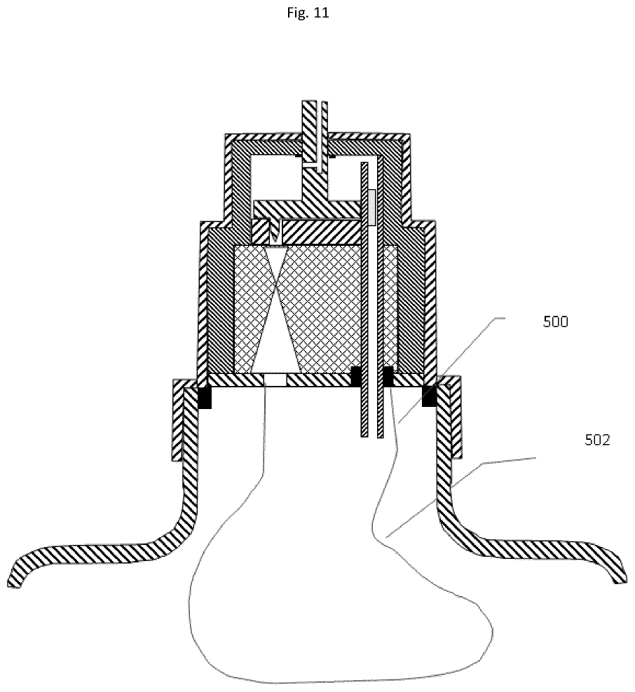

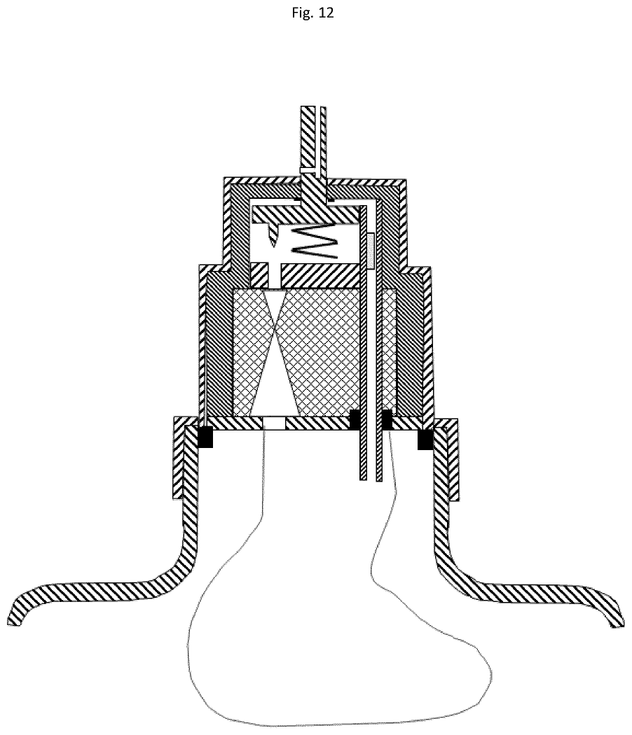

FIGS. 9-12 show schematically third and fourth embodiments of valve assemblies according to the present invention which are adapted for use with bag on valve (BOV) systems. In these embodiments of the invention the valve assembly is sealingly joined by the transversing wall to the neck 500 of a flexible container (often called a “bag”) 502 which is intended to contain the product. This flexible container is inserted into the rigid vessel before filling and the valve assembly connected to the neck 417 of the rigid vessel, by crimping, welding, gluing, using a threaded connection or the like after pressurised gas has been filled into the vessel. The bag subsequently can be filled with product via the bypass pathway.

For BoV systems where the product to be dispensed is inside a flexible bag within a pressurised gas vessel then preferably the maximum dispensing pressure is 900 kPa. Preferably the maximum cracking pressure of the flow control valve is 1000 kPa. Preferably, the maximum operating pressure inside the rigid vessel is 1100 kPa after filling and the maximum filling pressure is 1500 kPa.

More preferably the minimum dispensing pressure is 400 kPa. More preferably the minimum cracking pressure of the flow control valve is 100 kPa More preferably the minimum operating pressure inside the rigid vessel after filling is 600 kPa. More preferably the minimum filling pressure is 1000 kPa.

Even more preferably the dispensing pressure equal to or greater than 700 kPa and less than or equal to 900 kPa. Even more preferably the cracking pressure of the flow control valve equal to or greater than 100 kPa and less than or equal to 1050 kPa. Even more preferably the operating pressure inside the rigid vessel after filling is equal to or greater than 800 kPa and less than or equal to 1100 kPa.

Even more preferably the filling pressure is equal to or greater than 1000 kPa and less than or 1400 kPa.

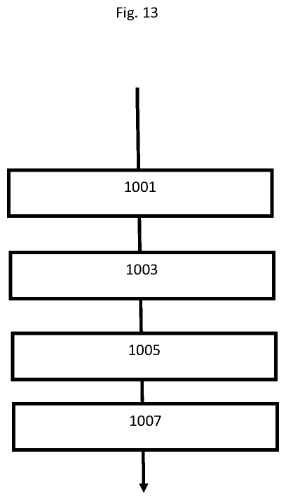

FIG. 13 illustrates the steps in a method for filling a vessel connected to a valve assembly according to the present invention, which method comprises the steps of:
1001 Connecting the open end of the valve stem to, or surrounding the valve stem with a cover connectable to, a source of high-pressure fluid (which may be a compressed gas, or compressed gas and product for pMDI systems, or a product for BoV systems) at a predetermined filling pressure;
1003 Moving the valve stem or providing a reverse flow pathway to the metering chamber so that there is a fluid communication pathway between the valve stem and the bypass pathway, flow control valve and interior of the vessel. In the case of the embodiments in which the bypass pathway extends through the valve stem then this may entail covering the transverse bore 438 in the valve stem, for example by surrounding it with the filling head or a separate cover, or depressing the valve stem so that the transverse bore is in the metering chamber. In the embodiments of the invention where the bypass pathway is not in the valve stem, then this can be achieved by depressing the valve stem so that the transverse bore is in the metering chamber so that the filling fluid can travel into the valve stem, leave the valve stem by the transverse bore, enter the metering chamber, flow to the inlet of the channel forming the bypass pathway, flow to the flow control valve, flow past the flow control valve and enter the vessel, or by forcing the filling fluid past the valve stem to atmosphere seal (which normally prevents pressurised fluid inside the valve assembly from exiting the valve assembly) in the reverse direction into the metering chamber;
1005 Allowing the high-pressure fluid to flow through said fluid communication pathway into the vessel for a predetermined length of time; and,
1007 Interrupting the supply of high-pressure fluid. Interrupting the supply of high-pressure fluid can be performed by disconnecting the filling head from the valve stem and/or by closing a valve between the high-pressure fluid supply and/or, if relevant, by raising the valve stem.

The valve stems of the valve assemblies of the present invention can be filled using prior art filling heads adapted as necessary to the dimensions of the valve stem. In order to enable rapid filling of the vessels to which the valve assemblies are attached, the flow resistance in the pathways used for filling should be minimised while at the same time the dispensing characteristics of the valve stem are preferably remain unchanged compared to prior art valve stems. Some conventional valve stems have a central bore and transverse bore diameter of 1.0 mm which restricts the filling speed to a certain rate but gives a satisfactory dispensing plume. Preferably in the embodiments of the invention in which the filling takes place though the central bore of a valve stem, the flow control arrangement is fitted as close to the second (internal) end of the valve stem as possible to allow a continuously large bypass pathway bore diameter for as long as possible. For example, it could be in the form of a duck-billed valve fitted onto the second end of the valve stem. If the valve stem has a diameter of 3.6 mm and the central bore has a diameter of 1.0 mm from the first end to the transverse bore, then the bore of the following bypass pathway could be 1.5 mm or 1.6 mm or more. This reduces the resistance to flow during filling and also provides a large diameter passageway for inserting the flow control arrangement into the valve stem.

If the flow control arrangement is positioned inside the bypass pathway then it may have a diameter which is slightly larger than that of the bypass pathway, for example 0.05 mm or 0.1 mm larger, so that the flow control arrangement can be inserted into the valve stem with an interference fit and thereby maintained in the desired position. In order to minimise the flow resistance further, the opening in the first end of the valve stem can be maintained at the desired size for dispensing with the correct plume, for example 1.0 mm, and the diameter of the central bore can increase in the direction towards the flow control arrangement, for example to 1.5 mm. The transverse bore can also have an increased diameter.

In the embodiments of the invention comprising a bypass pathway which does not extend though the valve stem, the channel in the bypass pathway can have a large diameter, preferably, equal to or greater than 1.5 mm, more preferably equal to or greater than 1,6 mm, yet more preferably equal to or greater than 1.7 mm, or more than 1.8 mm or 1.9 mm. Preferably the diameter is equal to or less than 2 mm in order to avoid increasing the diameter of the pressure control valve too much. Preferably the pressure control valve is fixed in the partition wall or below it. In order to reduce flow resistance, the diameter of the transverse bore in the valve stem can be equal to or greater than 1.5 mm.

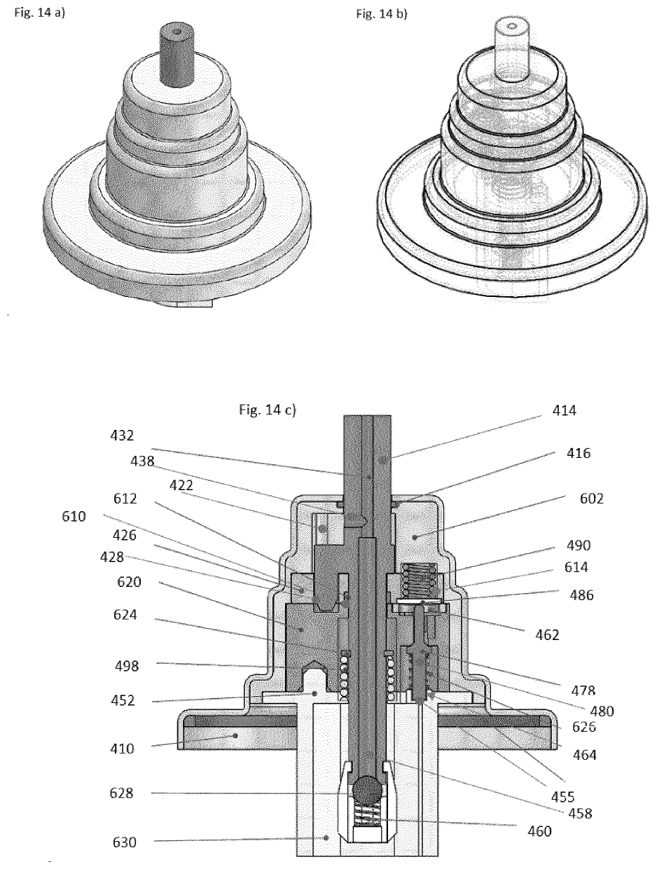

In a further embodiment of a bypass pathway, the bypass pathway extends through the wall of the valve body so that it has an inlet opening which is accessible from the exterior of the valve assembly. This permits the filling of an attached vessel without using the valve stem, as a filling head could be adapted to seal against the inlet opening and fill the vessel though the bore of the bypass pathway. FIG. 14 a) shows a perspective schematic view of an embodiment of a BoV valve assembly 400 for attachment to the inlet of a spray device vessel. FIG. 14b) shows the same view with internal components shown by dotted lines. FIG. 14c) shows a cross-section through the valve assembly of FIG. 14a) when seen from the side. In this valve assembly a bypass channel extends all the way through the valve stem and is intended to open out inside the vessel. The components of the valve assembly include a crimpable cap 410, preferably made of a soft metal such as aluminium which encloses and holds, preferably by interference fit or adhesive, a main housing 602, preferably made from an engineering polymer or the like which preferably has been cast or injection moulded.

The main body comprises an opening 604 for the valve stem 432 and a valve body to atmosphere seal 416 provides a fluid tight seal between the valve stem, main housing and cap. A metering chamber 422 is formed in the main housing and the base of the metering chamber is formed by a partition wall 426 which acts a metering chamber base 608 and which has a central through hole with seal 612 for the valve stem and a further through hole 614 for a poppet valve biasing means in the form of a compression spring 490 which is part of the pressure regulating valve. The though hole is closed at the lower end by a diaphragm 486 which is part of the pressure regulating valve and which is biased downwards by the regulating compression spring 490. The active surface of the diaphragm facing the spring 490 is A1. These components are preferably held in place by a valve housing 620 which has a passageway which extends from below the diaphragm to the entrance to metering chamber. The valve housing is held in place by an interference fit with the main housing 602 and/or by an interference fit with a transversing wall 452 mounted on or below the valve housing. Resilient means such a compression spring 498 which acts upon a locking ring 624 or the like on the valve stem biases the valve stem into the non-dispensing position are arranged inside the transversing wall and/or valve housing 620. The valve housing comprises a valve chamber which surrounds the poppet valve of 478 of the pressure regulating valve. Poppet valve can move towards and away from the diaphragm. When the valve assembly is open to atmosphere the regulating pressure spring pushes the diaphragm against the poppet valve so that it is in the open position. Optionally the poppet valve of can be loosely maintained in the valve open position by a weak tension spring 626 which pulls the poppet valve towards the transversing wall 452 and prevents the poppet valve moving into the closed position by the force of gravity when the spray device is inverted. The poppet valve has a collar 628 with an area A2 normal to the direction of movement of the poppet valve and an end surface with an area A3 normal to the direction of movement of the poppet valve.

Transversing wall 452 has a circular wall 630 which extends downwards, away from the valve housing, and is able to receive a bag containing the product concentrate.

The base of the valve stem comprises a sealing ball 628 which is biased onto a position to close the bypass pathway 458 by a bypass closing spring 468. The closing force of the bypass closing spring is chosen so that the bypass can be opened by applying high pressure gas to the opposite, open end of the valve stem.

The areas A1, A2 and A3 and the opposing spring force exerted by regulating compression spring 616 on the diaphragm are chosen so that the high pressure of the compressed gas in a vessel to which the valve assembly is attached is regulated down to the desired dispensing pressure. For example, if the starting pressure in the vessel is 10 bar (1000 kPa) and the desired dispensing pressure is 6.1 bar (610 KPa=0.61 N/mm²) then the poppet valve will close when the force exerted on the diaphragm and the areas of the poppet valve normal to the diaphragm by gas at 610 KPa overcomes the regulating compression spring force. So, if the area A1 of the diaphragm is 8.55mm², the area A2 of the collar of the poppet valve is 1.5 mm² and the area A3 of the end of the poppet valve is 0.95mm², then the force exerted against the regulating compression spring at 610 KPa is (8.55+1.5+0.95) mm²×0.61 N/mm²=11×0.61=0.67 N. If the movement of the diaphragm for open to closed is 1 mm, then the regulating compression spring should provide a spring force of 0.67 N/mm.

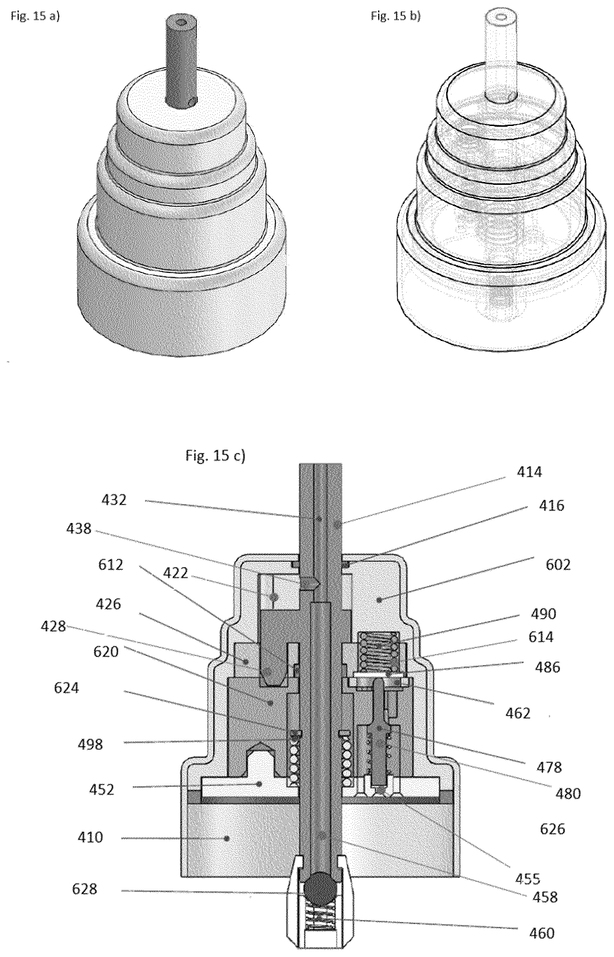

FIGS. 15a), 15b) and 15 c) show a corresponding pMDI valve assembly in which like components use the same reference numbers as used for the BoV valve assembly of FIG. 14a) to 14c). The two valve assemblies are substantially the same, except that the pMDI does not require a circular wall 630 to retain a bag. The pMDI may use a different vessel pressure and dispensing pressure than those used in the BoV example above.

The areas A1, A2 and A3 and the opposing spring force exerted by regulating compression spring 490 on the diaphragm are chosen so that the high pressure of the compressed gas in a vessel to which the valve assembly is attached is regulated down to the desired dispensing pressure. For example, if the starting pressure in the vessel is 16.5 bar (1650 kPa) and the desired dispensing pressure is 10.3 bar (1030 KPa=1.03 N/mm²) then the poppet valve will close when the force exerted on the diaphragm and the areas of the poppet valve normal to the diaphragm by gas at 1030 KPa overcomes the regulating compression spring force. So, if the area A1 of the diaphragm is 8.55 mm², the area A2 of the collar of the poppet valve is 1.5 mm² and the area A3 of the end of the poppet valve is 0.95 mm², then the force exerted against the regulating compression spring at 1030 KPa is (8.55+1.5+0.95) mm²×1.03 N/mm²=11×1.03=11.33 N. If the movement of the diaphragm for open to closed is 1 mm, then the regulating compression spring must provide a spring force of 11.33 N/mm.

Suitable dimensions for some components of the valve assembly are:
diaphragm diameter greater than or equal to 2 mm and less than or equal to 4 mm, and/or
metering chamber volume greater than or equal to 0.01 ml and less than or equal to 0.50 mm, and/or
bypass channel diameter greater than or equal to 0.5 mm and less than or equal to 1.5 mm.

Thus, the present invention relates to valve assemblies (400) for the dispensing of pressurised fluid comprising a valve body (402) with a metering chamber (422) connectable to a vessel with fluid to be dispensed wherein the valve assembly further comprises a pressure regulating valve and a bypass pathway (458) extending past the metering chamber and pressure regulating valve to allow filling of a vessel connected to a valve assembly, wherein said bypass pathway comprises a flow control arrangement (460) which allows one-way fluid flow from the surroundings in the direction towards and into the vessel.

A vessel and valve assembly arrangement comprises a vessel and a valve assembly (400) for the dispensing of pressurised fluid from the vessel, wherein said valve assembly comprises a valve body (402) with a metering chamber (422) fluidly connectable via a pressure regulating valve to the neck of the vessel wherein the valve assembly further comprises a bypass pathway (458) extending past the metering chamber and pressure regulating valve to the interior of the vessel to allow filling of the vessel attached to a valve assembly, wherein said bypass pathway comprises a flow control arrangement (460) which allows one-way fluid flow from the surroundings in the direction towards and into the vessel.

1. A valve assembly (400) for the dispensing of pressurised fluid from a vessel used in an spray device which is intended to provide a metered dose of an active substance to the mouth of a user comprising:
a valve body (402) with a metering chamber (422) with a sealable fluid dispensing through hole (428) in fluid communication with an inlet passage (455) in a transversing wall (452) which extends across the cross section of the valve body, and a movable valve stem (414) comprising a transverse bore (438) in fluid communication with a longitudinal bore (432),
wherein said valve stem (414) is adapted to be movable between a closed position, where it closes said sealable fluid dispensing through hole (428) and opens fluid communication between the metering chamber (422) and the surroundings via said transverse bore (438) and longitudinal bore (432), and an open position, where it opens said fluid dispensing through hole (428) and closes fluid communication via said transverse bore (438) and longitudinal bore (432) between said metering chamber (422) and the surroundings,
characterised in that it comprises a pressure regulating valve (462) with a high-pressure fluid inlet (464) in fluid communication with said inlet passage (455) in said transversing wall (452) and a regulated pressure outlet (466) in fluid communication with the sealable fluid dispensing through hole (428) of the metering chamber (422);
and the valve assembly further comprises a bypass pathway (458) extending past the metering chamber (422) and the pressure regulating valve (462), and through the transverse wall (452) wherein said bypass pathway (459 comprises a flow control arrangement (460) which allows one-way fluid flow from the surroundings in the direction towards and through the transversing wall (452).

2. The valve assembly according to claim 1, characterised in that said bypass pathway (458) is comprised within said valve stem (414) or that said bypass pathway (458) is formed as a channel (466) which extends from the metering chamber (422) to, or through, the transversing wall (452) or that said bypass pathway (458) extends from outside the valve body (402) to, or through, said transversing wall (452).

3. The valve assembly according to claim 1 wherein the bypass pathway (458) comprises a duckbill one-way valve or a ball valve.

4. The valve assembly according to claim 1 characterised in that said pressure regulating valve (462) comprises a poppet valve (478) with a valve stem (480) which is biased into an open position by a force-producing valve biasing means (490) which is arranged to bias the poppet valve in the direction towards the high-pressure fluid inlet (464).

5. The valve assembly according to claim 4 characterised in that said valve biasing means (490) is a block of rubber-like, gel or sponge-like resilient material, or a spring acting on a diaphragm.

6. A vessel and valve assembly arrangement comprising a vessel with a neck (417; 500) and a valve assembly (400) according to claim 1, characterised in that said valve assembly is connected to the neck of the vessel and the bypass pathway (458) extends through said neck into the interior of the vessel.

7. The vessel and valve assembly arrangement according to claim 6 wherein said bypass pathway (458) extends into a bag (502) in the interior of the vessel.

8. The vessel and valve assembly arrangement according to claim 6 wherein the vessel contains compressed gas.

9. A method of filling a vessel comprising a valve assembly according to claim 1 comprising the steps of:
(1001) Connecting the open end of the valve stem to, or surrounding the valve stem with a cover connectable to, a source of high-pressure fluid at a predetermined filling pressure;
(1003) Moving the valve stem, or providing reverse flow pathway to the metering chamber, so that there is a fluid communication pathway between the valve stem and the bypass pathway, flow control valve and interior of the vessel;
(1005) Allowing the high-pressure fluid to flow through said fluid communication pathway into the vessel for a predetermined length of time; and,
(1007) Interrupting the supply of high-pressure fluid.
